(19) United States
(12) Patent Application Publication
(10) Pub. No.: US 2026/0092908 A1
(43) Pub. Date: Apr. 2, 2026

(54) METHOD, EQUIPMENT, ELECTRONIC DEVICE AND READABLE STORAGE MEDIUM FOR TOXIC GAS DETECTION

(71) Applicant: JINAN BENAN TECHNOLOGY DEVELOPMENT CO., LTD., Jinan (CN)

(72) Inventors: Yunjiang QI, Jinan (CN); Zhigang SUN, Jinan (CN); Tengteng SUN, Jinan (CN); Honghai ZHANG, Jinan (CN); Chuanliang ZHANG, Jinan (CN); Yongjun MA, Jinan (CN)

(21) Appl. No.: 19/412,979

(22) Filed: Dec. 9, 2025

Related U.S. Application Data
(63) Continuation of application No. PCT/CN2024/089854, filed on Apr. 25, 2024.

(51) Int. Cl. G01N 33/00; G01C 21/20

(52) U.S. Cl. CPC G01N 33/0075; G01C 21/20; G01N 33/0068

(57) ABSTRACT

An equipment and a method for toxic gas detection include acquiring current toxic gas components in a target environment, a current toxic gas concentration of each of the current toxic gas components at each detection position, and first position identification information at each detection position; determining a current diffusion position based on the current toxic gas components, and the current toxic gas concentration and the first position identification information at each detection position; and acquiring second position identification information of the person, and determining and outputting a current escape route based on the current diffusion position and the second position identification information.

### CROSS-REFERENCE TO RELATED APPLICATION

This application is a continuation of PCT application serial no. PCT/CN2024/089854, filed on Apr. 25, 2024, which claims the priority and benefit of Chinese patent application serial no. 202311050402.8, filed on Aug. 21, 2023. The entirety of PCT application serial no. PCT/CN2024/089854 and Chinese patent application serial no. 202311050402.8 are hereby incorporated by reference herein and made a part of this specification.

### TECHNICAL FIELD

The present application relates to the technical field of toxic gas detection, and in particular relates to a method, equipment, electronic device and readable storage medium for toxic gas detection.

### BACKGROUND ART

In a production process, when toxic gas exists in the environment, the toxic gas will threaten physical health of workers; the higher the content of the toxic gas, the greater the impact on the human body. Therefore, it is necessary to detect the environment to determine whether the toxic gas exists and the content of the toxic gas. Toxic gas detection devices commonly used in industry usually use electrochemical sensors to detect the toxic gases. A working principle of the toxic gas detection devices is to measure gas concentration by reacting with the detected gas and generating an electrical signal proportional to the gas concentration. When the concentration of the toxic gas in a certain environment exceeds a specific threshold, the environment is deemed to have excessive toxic gas, and the workers in the environment need to leave the environment immediately.

When a toxic gas leakage is detected in the environment, an alarm signal is issued, an alarm does not have direction-guiding capability, and the workers in the environment may panic upon hearing the alarm, making it difficult for the workers to determine an escape route in a timely manner. Therefore, how to timely determine the escape route when a toxic gas leak occurs has becoming increasingly important.

### SUMMARY

In order to timely determine an escape route in case of toxic gas leakage occurs, the present application provides a method, equipment, electronic device and readable storage medium for toxic gas detection.

The above-mentioned objective of the present application is achieved by the following technical solutions:

In a first aspect, a method for toxic gas detection is provided, which includes:
acquiring current toxic gas components in a target environment, a current toxic gas concentration of each of the current toxic gas components at each detection position, and first position identification information at each detection position;
determining a current diffusion position based on the current toxic gas components, and the current toxic gas concentration and the first position identification information at each detection position; and
acquiring second position identification information of the person, and determining and outputting a current escape route based on the current diffusion position and the second position identification information.

By adopting the above technical solution, the current toxic gas components in the target environment, the current toxic gas concentrations corresponding to the current toxic gas components at each detection position, and the first position identification information corresponding to each detection position are acquired; the current diffusion position is determined based on the current toxic gas components, and the current toxic gas concentrations and the first position identification information corresponding to each detection position; when determining the escape route, it is necessary to avoid the diffusion position of the toxic gas; the second position identification information corresponding to the person is acquired; and the current escape route is accurately determined and output based on the current diffusion position and the second position identification information to guide the person in the target environment to escape, and timely determine the escape route of the person in the target environment in case of toxic gas leakage.

In a possible implementation, before determining a current diffusion position based on the current toxic gas components, and the current toxic gas concentration and the first position identification information at each detection position, the method further includes:
determining whether the target environment is a dangerous area based on the current toxic gas concentration at each detection position;
acquiring an environmental image of the target environment, when the target environment is the dangerous area, and determining whether there is a person in the target environment based on the environmental image.

In another possible implementation, the determining whether the target environment is the dangerous area based on the current toxic gas concentrations corresponding to each detection position includes:
acquiring a first relationship curve corresponding to each detection position, wherein the first relationship curve is configured to characterize a relationship between a historical toxic gas concentration and historical time corresponding to each detection position within the historical time;
determining the environmental concentration threshold corresponding to each detection position based on the first relationship curve; wherein
when the current toxic gas concentration is greater than the environmental concentration threshold, determining the detection position corresponding to the current toxic gas concentration as a dangerous position; and
when a number of dangerous positions is greater than the threshold, the target environment is the dangerous area.

In another possible implementation, before determining the current diffusion position based on the current toxic gas components, and the current toxic gas concentrations and the first position identification information corresponding to each detection position, the method further includes:
determining the current diffusion direction corresponding to the current toxic gas components based on the current toxic gas components, a preset toxic gas component, a preset diffusion direction, and a diffusion relationship between the preset toxic gas component and the preset diffusion direction;
determining the current toxic gas with highest concentration from the current toxic gas concentrations corresponding to each detection position, and determining the detection position corresponding to the current toxic gas with highest concentration as the first detection position; and
determining the current diffusion position based on the current diffusion direction and the first position identification information corresponding to the first detection position.

In another possible implementation, the determining the current escape route based on the current diffusion position and the second position identification information includes:
determining a diffusion range based on the current toxic gas concentrations and the first position identification information corresponding to each detection position;
determining the current escape route based on the current diffusion position, the second position identification information, and the diffusion range.

In another possible implementation, the determining the diffusion range based on the current toxic gas concentrations and the first position identification information corresponding to each detection position includes:
determining a current minimum toxic gas concentration from the current toxic gas concentrations corresponding to each detection position based on environmental concentration thresholds corresponding to each detection position, and determining a detection position corresponding to the current minimum toxic gas concentration as a second detection position, wherein the current minimum toxic gas concentration is higher than the environmental concentration threshold corresponding to the second detection position;
determining a diffusion distance based on the first position identification information corresponding to the first detection position and the first position identification information corresponding to the second detection position; and
determining the diffusion range based on the first position identification information corresponding to the first detection position, the first position identification information corresponding to the second detection position, and the diffusion distance.

In another possible implementation, when the current toxic gas component is at least in a number of two, the method further includes:
when there is no person in the target environment, determining a current danger level corresponding to each current toxic gas component based on each current toxic gas component, the preset toxic gas component, a preset danger level, and the relationship between the preset toxic gas component and the preset danger level;
determining a component concentration corresponding to each current toxic gas component based on the current toxic gas concentration corresponding to each current toxic gas component at each detection position;
determining a current emergency toxic gas component from each current toxic gas component based on the current danger level and the component concentration corresponding to each current toxic gas component;
acquiring historical toxic gas components, historical gas treatment measures, and a treatment relationship between the historical toxic gas components and the historical gas treatment measures; and
determining current gas treatment measures based on the current emergency toxic gas components, the historical toxic gas components, the historical gas treatment measures, and the treatment relationship.

In a second aspect, an equipment for toxic gas detection is provided, the equipment including:
a first acquisition module configured to acquire current toxic gas components in a target environment, current toxic gas concentrations corresponding to the current toxic gas components at each detection position, and first position identification information corresponding to each detection position;
a first determination module configured to determine a current diffusion position based on the current toxic gas components and the current toxic gas concentrations and the first position identification information corresponding to each detection position; and
a second determination module configured to acquire second position identification information corresponding to a person, and determine and output a current escape route based on the current diffusion position and the second position identification information.

In a possible implementation, the equipment further includes: a first judging module and a second judging module, wherein
the first judging module is configured to judge whether the target environment is a dangerous area based on the current toxic gas concentrations corresponding to each detection position; and
the second judging module is configured to, when the target environment is the dangerous area, acquire an environmental image corresponding to the target environment, and judge whether there is the person in the target environment based on the environmental image.

In another possible implementation, the first judging module, when judging whether the target environment is the dangerous area based on the current toxic gas concentrations corresponding to each detection position, is specifically configured to:
acquire a first relationship curve corresponding to each detection position, wherein the first relationship curve is configured to characterize a relationship between a historical toxic gas concentration and historical time corresponding to each detection position within the historical time;
determine the environmental concentration thresholds corresponding to each detection position based on the first relationship curve;
when the current toxic gas concentration is greater than the environmental concentration threshold, determine the detection position corresponding to the current toxic gas concentration as a dangerous position; and
when a number of the dangerous positions is greater than the threshold, the target environment is the dangerous area.

In another possible implementation, the first determination module, when determining the current diffusion position based on the current toxic gas components, and the current toxic gas concentrations and the first position identification information corresponding to each detection position, is specifically configured to:
determine a current diffusion direction corresponding to the current toxic gas components based on the current toxic gas components, a preset toxic gas component, a preset diffusion direction, and a diffusion relationship between the preset toxic gas component and the preset diffusion direction;
determine the current toxic gas with highest concentration from the current toxic gas concentrations corresponding to each detection position, and determine the detection position corresponding to the current toxic gas with highest concentration as the first detection position; and
determine the current diffusion position based on the current diffusion direction and the first position identification information corresponding to the first detection position.

In another possible implementation, the second determination module, when determining the current escape route based on the current diffusion position and the second position identification information, is specifically configured to
determine a diffusion range based on the current toxic gas concentrations and the first position identification information corresponding to each detection position;
determine the current escape route based on the current diffusion position, the second position identification information, and the diffusion range.

In another possible implementation, the second determination module, when determining the diffusion range based on the current toxic gas concentrations and the first position identification information corresponding to each detection position, is specifically configured to
determine an current minimum toxic gas concentration from the current toxic gas concentrations corresponding to each detection position based on environmental concentration thresholds corresponding to each detection position, and determine a detection position corresponding to the current minimum toxic gas concentration as a second detection position, where the current minimum toxic gas concentration is higher than the environmental concentration threshold corresponding to the second detection position;
determine a diffusion distance based on the first position identification information corresponding to the first detection position and the first position identification information corresponding to the second detection position; and
determine the diffusion range based on the first position identification information corresponding to the first detection position, the first position identification information corresponding to the second detection position, and the diffusion distance.

In another possible implementation, the equipment further includes: a second acquisition module, a third determination module, a calculating module, and an outputting module, where
in another possible implementation, when the current toxic gas component is at least in a number of two, the equipment further includes: a fourth determination module, a fifth determination module, a sixth determination module, a third acquisition module, and a seventh determination module, wherein,
the fourth determination module is configured to, when there is no person in the target environment, determine a current danger level corresponding to each current toxic gas component based on each current toxic gas component, the preset toxic gas component, the preset danger level, and the relationship between the preset toxic gas component and the preset danger level;
the fifth determination module is configured to determine a component concentration corresponding to each current toxic gas component based on the current toxic gas concentration corresponding to each current toxic gas component at each detection position;
the sixth determination module is configured to determine the current emergency toxic gas component from each current toxic gas component based on the current danger level and the component concentration corresponding to each current toxic gas component;
the third acquisition module is configured to acquire historical toxic gas components, historical gas treatment measures, and a treatment relationship between the historical toxic gas components and the historical gas treatment measures; and
the seventh determination module is configured to determine the current gas treatment measures based on the current emergency toxic gas components, the historical toxic gas components, the historical gas treatment measures, and the treatment relationship.

In a third aspect, an electronic device is provided, which includes:
one or more processors;
a memory;
one or more application programs, where the one or more application programs are stored in the memory and configured to be executed by the one or more processors to implement operations corresponding to the method for toxic gas detection according to any possible implementation of the first aspect.

In a fourth aspect, a computer-readable storage medium is provided, wherein the storage medium stores at least one instruction, at least one program, code set or instruction set, and the at least one instruction, at least one program, code set or instruction set is loaded and executed by a processor to implement the method for toxic gas detection according to any possible implementation of the first aspect.

In summary, the present application includes at least one of the following beneficial technical effects:

The present application provides a method, equipment, electronic device and readable storage medium for toxic gas detection, compared with the related art, in the present application, by acquiring the current toxic gas components in the target environment, the current toxic gas concentration corresponding to the current toxic gas components at each detection position, and the first position identification information corresponding to each detection position respectively; the current diffusion position is determined based on the current toxic gas components, and the current toxic gas concentrations and the first position identification information corresponding to each detection position; when determining the escape route, it is necessary to avoid the diffusion position of the toxic gas; the second position identification information corresponding to the person is acquired; and the current escape route is accurately determined and output based on the current diffusion position and the second position identification information to guide the person in the target environment to escape, and timely determine the escape route of the person in the target environment in case of toxic gas leakage.

### BRIEF DESCRIPTION OF THE DRAWINGS

FIG. 1 is a schematic flowchart of a method for toxic gas detection according to an embodiment of the present application.

FIG. 2 is a schematic structure diagram of an equipment for toxic gas detection according to an embodiment of the present application.

FIG. 3 is a schematic structure diagram of an electronic device according to an embodiment of the present application.

### DETAILED DESCRIPTION

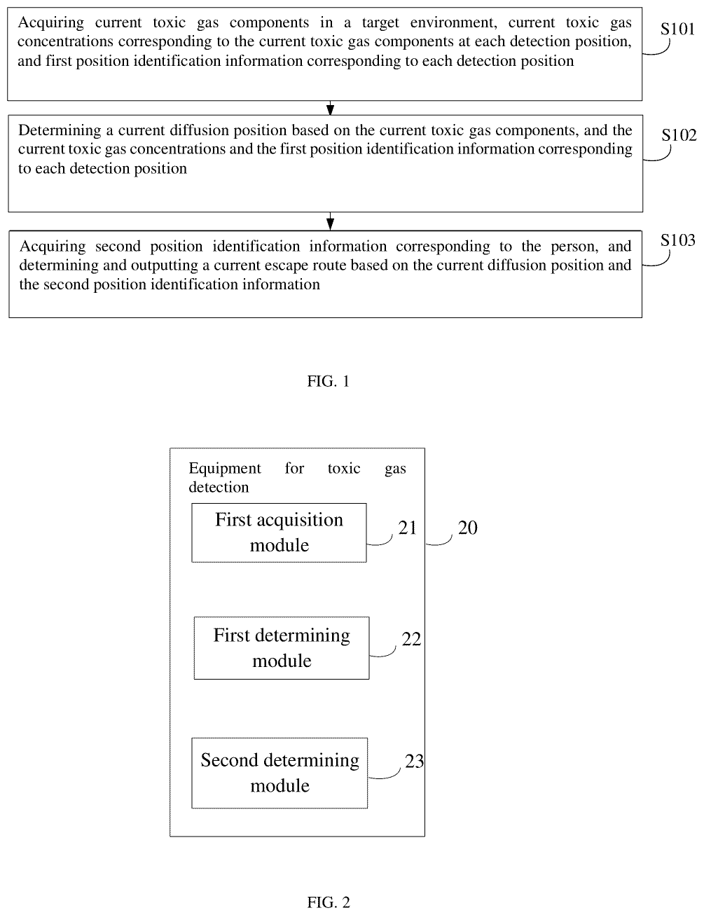

The present application is described in further detail below with reference to FIGS. 1-3.

This specific embodiment is merely an explanation of the present application and does not constitute a limitation to the present application. Those skilled in the art may make modifications to this embodiment that do not involve creative contributions as needed after reading this specification, but all such modifications shall be protected by patent law as long as they fall within the scope of the claims of the present application.

In order to make the objectives, technical solutions, and advantages of the embodiments of the present application clearer, the technical solutions in the embodiments of the present application will be described clearly and completely below in conjunction with the accompanying drawings in the embodiments of the present application. Apparently, the described embodiments are only a part of the embodiments of the present application, not all the embodiments. Based on the embodiments of the present application, all other embodiments obtained by those of ordinary skilled in the art without creative work shall fall within the scope required to be protected by the claims of the present application.

In addition, the term “and/or” described herein only describes a relationship among associated objects, and denotes that there may be three relationships. For example, A and/or B may denote three situations that there is A alone; there are A and B at the same time; and there is B alone. In addition, the character “/” described herein generally denotes that forward and backward associated objects are in an “or” relationship unless otherwise noted.

The embodiments of the present application will be described in further detail below referring to the accompanying drawings in the description.

The embodiment of the present application provides a method for toxic gas detection, executed by an electronic device, which may be a server or a terminal device, wherein, the server may be an independent physical server, a server cluster or a distributed system composed of a plurality of physical servers, or a cloud server providing cloud computing services. The terminal device may be a smartphone, a tablet computer, a laptop computer, a desktop computer, etc., but is not limited thereto. The terminal device and the server may be directly or indirectly connected through wired or wireless communication, which is not limited in the embodiment of the present application, wherein, referring to FIG. 1, the method may include:
step S101. acquiring current toxic gas components in a target environment, current toxic gas concentrations corresponding to the current toxic gas components at each detection position, and first position identification information corresponding to each detection position respectively.

For the embodiment of the present application, the target environment may be a closed environment, in which at least two detection positions are arranged, and at least one toxic gas detector is provided at each detection position. The toxic gas detector may acquire in real time the current toxic gas components in the target environment and the current toxic gas concentrations corresponding to the current toxic gas components at each detection position, or acquire the current toxic gas components in the target environment and the current toxic gas concentrations corresponding to the current toxic gas components at each detection position at intervals of a preset time, or acquire the current toxic gas components in the target environment and the current toxic gas concentrations corresponding to the current toxic gas components at each detection position when detecting an acquisition instruction triggered by a user, which is not limited in the embodiment of the present application.

For the embodiment of the present application, the electronic device may acquire from the toxic gas detector in real time the current toxic gas components in the target environment and the current toxic gas concentrations corresponding to the current toxic gas components at each detection position, or acquire from the toxic gas detector the current toxic gas components in the target environment and the current toxic gas concentrations corresponding to the current toxic gas components at each detection position at intervals of the preset time, or acquire the current toxic gas components in the target environment and the current toxic gas concentrations corresponding to the current toxic gas components at each detection position when detecting a trigger instruction from the user, which is not limited in the embodiment of the present application.

It should be noted that the toxic gas detector may be a device independent of the electronic device.

For the embodiment of the present application, when the current toxic gas concentrations in the target environment exceed a standard and there is a person in the target environment, it is also necessary to acquire the first position identification information corresponding to each detection position, which may be acquired from local storage, or from other devices, or the first position identification information corresponding to each detection position input by the user, which is not limited in the embodiment of the present application.

For the embodiment of the present application, the first position identification information of the detection positions corresponding to the detection positions may be coordinates or labels of the toxic gas detector.

In the above embodiment of the present application, after acquiring the current toxic gas components in the target environment, the current toxic gas concentrations corresponding to the current toxic gas components at each detection position, and the first position identification information corresponding to each detection position, a display may display in real time the current toxic gas components in the target environment, the current toxic gas concentrations corresponding to the current toxic gas components at each detection position, and the first position identification information corresponding to each detection position, or display the current toxic gas components in the target environment, the current toxic gas concentrations corresponding to the current toxic gas components at each detection position, and the first position identification information corresponding to each detection position when detecting a display instruction triggered by the user, so that the user can grasp leakage of harmful gases in the target environment in real time.
Step S102. determining a current diffusion position based on the current toxic gas components, and the current toxic gas concentrations and the first position identification information corresponding to each detection position.

For the embodiment of the present application, after acquiring the first position identification information corresponding to each detection position, it is necessary to determine the escape route for the person. When determining the escape route, it is necessary to determine characteristics of the current toxic gas according to the current toxic gas components. The characteristics of the current toxic gas may be density of the current toxic gas. For example, when the density of the toxic gas is greater than that of air, the toxic gas is closer to the surface of Earth; when the density of the toxic gas is less than that of air, the toxic gas tends to diffuse upward, and the escape route needs to escape to lower places in the target environment. The current diffusion position of the current toxic gas components is determined according to distribution of the current toxic gas concentrations at each detection position and the first position identification information of the detection positions. The current diffusion position may be the detection position corresponding to the highest current toxic gas concentration.
Step S103, acquiring second position identification information corresponding to the person, and determining and outputting a current escape route based on the current diffusion position and the second position identification information.

For the embodiment of the present application, the second position identification information may be coordinates or a relative position of a certain detection position. The first position identification information corresponding to the person may be acquired in real time, or acquired at intervals of specific time, or acquired when detecting the acquisition instruction triggered by the user, which is not limited in the embodiment of the present application.

For the embodiment of the present application, after determining a current diffusion direction of the current toxic gas components, the first position identification information where the person is located is determined as a starting position of the escape route, and an escape direction for the person is determined based on the current diffusion position. The escape direction for the person should avoid the current diffusion position. The escape route is determined jointly based on the starting position and the current diffusion position, and the escape route may be output in a form of audio when outputting the escape route.

The embodiment of the present application provides a method for toxic gas detection, compared with the related art, in the embodiment of the present application, by acquiring the current toxic gas components in the target environment, the current toxic gas concentrations corresponding to the current toxic gas components at each detection position, and the first position identification information corresponding to each detection position respectively; the current diffusion position of the toxic gas is determined based on the current toxic gas components, and the current toxic gas concentrations and the first position identification information corresponding to each detection position; when determining the escape route, it is necessary to avoid the diffusion position of the toxic gas; the second position identification information corresponding to the person is acquired; and the current escape route is accurately determined and output based on the current diffusion position and the second position identification information to guide the person in the target environment to escape, and timely determine the escape route for the person in the target environment in case of toxic gas leakage.

In a possible implementation of the embodiment of the present application, before determining the current diffusion position based on the current toxic gas components, and the current toxic gas concentrations and the first position identification information corresponding to each detection position, the method further includes: it is determined whether the target environment is a dangerous area based on the current toxic gas concentrations corresponding to each detection position respectively; and when the target environment is the dangerous area, environmental images corresponding to the target environment are acquired, and it is determined whether there is the person in the target environment based on the environmental images. In the embodiment of the present application, it is necessary to determine the escape route after determining that the target environment is the dangerous area and that there is the person in the target environment.

For the embodiment of the present application, a threshold for the target environment to reach the dangerous area may be determined based on a historical toxic gas concentration of the detection position, or a standard threshold may be directly used to compare with the current toxic gas concentrations to determine whether the current toxic gas concentration at the detection position exceeds the standard. For example, based on the historical toxic gas concentration of detection position one within one month, the threshold for reaching the dangerous area is determined to be 30 mg/m³, and the current toxic gas concentration is 25 mg/m³, then the current toxic gas concentration at the detection position one does not exceed the standard; for another example, the standard threshold is 10 mg/m³, and the current toxic gas concentration at the detection position one is 25 mg/m³, then the current toxic gas concentration at the detection position one exceeds the standard. It is determined whether the target environment is the dangerous area according to a number of detection positions where the current toxic gas concentration exceeds the standard, or the target environment is the dangerous area when there are detection positions in the target environment where the current toxic gas concentration exceeds the standard. For example, there are detection position one, detection position two, detection position three, detection position four, and detection position five in the target environment, and the current toxic gas concentrations at the detection position one, the detection position two, and the detection position three all exceed the standard, and a number threshold is 2, then the target environment is the dangerous area.

For the embodiment of the present application, by acquiring the environmental images corresponding to the target environment, the environmental images may be the environmental images corresponding to each position in the target environment, and the environmental images may be identified through a trained person identification model to determine whether there is the person. The trained person identification model may be trained based on each preset image dataset.

For the embodiment of the present application, it is determined whether the target environment is the dangerous area based on the current toxic gas concentrations of the target environment, and it is accurately determined whether there is the person through the environmental images of the target environment. When the target environment is the dangerous area and there is the person, the escape route is determined to reduce operating resources.

In another possible implementation of the embodiment of the present application, determining whether the target environment is the dangerous area based on the current toxic gas concentrations corresponding to each detection position may specifically include: a first relationship curve corresponding to each detection position is acquired; environmental concentration thresholds corresponding to each detection position is determined based on the first relationship curve; when the current toxic gas concentration is greater than the environmental concentration threshold, the detection position corresponding to the current toxic gas concentration is determined as a dangerous position; when a number of dangerous positions is greater than the threshold, the target environment is the dangerous area; when a number of the dangerous positions is not greater than the threshold, the target environment is not the dangerous area. In the embodiment of the present application, standards for excessive toxic gas content at each detection position may be different, and comparing with the current toxic gas concentrations at each detection position through a unified standard may lead to a wrong judgment result. For example, when the content of toxic gas A in position A exceeds 30 mg/m³, the position A is the dangerous position; when the content of toxic gas A in position B exceeds 10 mg/m³, the position B is the dangerous position. When the number of the dangerous positions in the target environment is greater than the threshold, the target environment is the dangerous area; when the number of the dangerous positions in the target environment is not greater than the threshold, the target environment is not the dangerous area.

The first relationship curve is used to characterize a relationship between the historical toxic gas concentration and historical time corresponding to each detection position within the historical time.

For the embodiment of the present application, the first relationship curve is a change of the historical toxic gas concentration of the detection position within the historical time, and respective thresholds when the toxic gas concentrations at each detection position exceed the standard are determined through the first relationship curve. For example, through the first relationship curve, the environmental concentration threshold of the detection position is determined to be 25 mg/m³. The environmental concentration thresholds corresponding to each detection position may all be the same, or partially the same. The current toxic gas concentration of the detection position is compared with the environmental concentration threshold of the detection position to determine whether the current toxic gas concentration of the detection position is the dangerous position, and it is determined whether the target environment is the dangerous area through the number of the dangerous positions.

For the embodiment of the present application, through the change in the historical toxic gas concentrations at each detection position in the target environment within the historical period, the environmental concentration thresholds corresponding to each detection position when the toxic gas concentrations exceed the standard are accurately determined, the current toxic gas concentrations at each detection position are compared with the corresponding environmental concentration thresholds to determine the dangerous positions, and it is accurately determined whether the target environment is the dangerous area according to the number of the dangerous positions.

In another possible implementation of the embodiment of the present application, the determining the current diffusion position based on the current toxic gas components, and the current toxic gas concentrations and the first position identification information corresponding to each detection position may specifically include: the current diffusion direction corresponding to the current toxic gas components is determined based on the current toxic gas components, a preset toxic gas component, a preset diffusion direction, and a diffusion relationship between the preset toxic gas component and the preset diffusion direction; the highest current toxic gas concentration is determined from the current toxic gas concentrations corresponding to each detection position, and the detection position corresponding to the highest current toxic gas concentration is determined as a first detection position; and the current diffusion position is determined based on the current diffusion direction and the first position identification information corresponding to the first detection position. In the embodiment of the present application, different toxic gas components correspond to different diffusion directions. Through the preset toxic gas component, the preset toxic gas component that is the same as the current toxic gas component is determined, and the current diffusion direction corresponding to the current toxic gas component is determined from the preset diffusion directions based on the diffusion relationship between the preset toxic gas component and the preset diffusion direction. In order to accurately determine the current diffusion position of the current toxic gas components, the detection position with the highest current toxic gas concentration is determined, i.e., the first detection position, which is closest to the location of toxic gas leakage, and the current diffusion position is determined through the first position identification information corresponding to the first detection position and the current diffusion direction.

For the embodiment of the present application, through a matching of the current toxic gas component and the preset toxic gas component, based on the diffusion relationship between the preset toxic gas component and the preset diffusion direction, the current diffusion direction among the preset diffusion directions is quickly determined, and the first position identification information of the first detection position of the leakage of the current toxic gas component is determined through the current toxic gas concentrations and the first position identification information corresponding to each detection position, and the current diffusion position is accurately determined according to the first position identification information of the first detection position and the current diffusion direction.

Specifically, the determining the current escape route based on the current diffusion position and the second position identification information may specifically include: a diffusion range is determined based on the current toxic gas concentrations and the first position identification information corresponding to each detection position; the current escape route is determined based on the current diffusion position, the second position identification information and the diffusion range. In the embodiment of the present application, a variation law of the current toxic gas is determined through the current toxic gas concentrations corresponding to each detection position, and the diffusion range of the current toxic gas concentrations is determined based on the first position identification information corresponding to each detection position. For example, the current toxic gas concentration at the detection position one is 50 mg/m³, and the current toxic gas concentration at the detection position two is 40 mg/m³, then the diffusion range is the range from the detection position one to the detection position two. An escapable range is determined according to the current diffusion direction and the diffusion range. The escapable range needs to avoid the current diffusion direction and the diffusion range. The second position identification information is determined as a starting point of the current escape route, and the escape route is determined based on the starting point of the current escape route and the escapable range. The current diffusion positions may include a upper area or a lower area and the location of the toxic gas leakage. When the target environment includes upper and lower floors, it is determined that the person escapes to lower or higher places through the current diffusion direction, and the escape route is determined based on the current diffusion direction and the diffusion range, that is, the escape route is opposite to the current diffusion position and the diffusion range.

For the embodiment of the present application, the diffusion range of the toxic gas is determined through the first position identification information and the current toxic gas concentrations of each detection position, the current diffusion direction and the diffusion range are avoided when determining the escape route, and the escape route is accurately determined based on the second position identification information.

Specifically, the determining the diffusion range based on the current toxic gas concentrations and the first position identification information corresponding to each detection position may specifically include: current minimum toxic gas concentration is determined from the current toxic gas concentrations corresponding to each detection position based on the environmental concentration thresholds corresponding to each detection position, and the detection position corresponding to the current minimum toxic gas concentration is determined as a second detection position; a diffusion distance is determined based on the first position identification information corresponding to the first detection position and the first position identification information corresponding to the second detection position; and the diffusion range is determined based on the first position identification information corresponding to the first detection position and the diffusion distance. In the embodiment of the present application, in order to accurately determine the diffusion range of the current toxic gas components, the detection positions corresponding to the current toxic gas concentrations greater than the environmental concentration threshold are all within the diffusion range of the current toxic gas, and the detection position with the highest current toxic gas concentration is a toxic gas diffusion center point, i.e., the first detection position, and the detection position corresponding to the current toxic gas concentration that is higher than the environmental concentration threshold and has the smallest difference is a diffusion edge point, i.e., the second detection position, and the diffusion range is determined through the first position identification information of the first detection position and the first position identification information of the second detection position.

Wherein, the minimum current toxic gas concentration is higher than the environmental concentration threshold corresponding to the second detection position.

For the embodiment of the present application, the first position identification information of the first detection position may be determined as a center point, a distance between the first detection position and the second detection position is determined based on the first position identification information of the first detection position and the first position identification information of the second detection position, and a circle is drawn with the distance between the first detection position and the second detection position as a radius, and the range covered by the circle is the current diffusion range. For example, the current toxic gas concentration corresponding to the detection position one is 50 mg/m³, the first position identification information is coordinate one, the current toxic gas concentration corresponding to the detection position two is 33 mg/m³, the first position identification information is coordinate two, the current toxic gas concentration corresponding to the detection position three is 20 mg/m³, the first position identification information is coordinate three, and the environmental concentration threshold is 28 mg/m³; then the coordinate one is the center point, and a difference between the coordinate one and the coordinate two is the distance between the detection position one and the detection position two.

For the embodiment of the present application, the diffusion range of the current toxic gas component is accurately determined through the changes in the current toxic gas concentration at each detection position and the first position identification information of each detection position.

In another possible implementation of the embodiment of the present application, the method may further include: when there is no person in the target environment, a current danger level corresponding to each current toxic gas component is determined based on each current toxic gas component, the preset toxic gas component, a preset danger level, and the relationship between the preset toxic gas component and the preset danger level; a component concentration corresponding to each current toxic gas component is determined based on the current toxic gas concentrations corresponding to each current toxic gas component at each detection position; a current emergency toxic gas component is determined from each current toxic gas component based on the current danger level and the component concentration corresponding to each current toxic gas component; historical toxic gas components, historical gas treatment measures, and a treatment relationship between the historical toxic gas components and the historical gas treatment measures are acquired; and current gas treatment measures are determined based on the current emergency toxic gas components, the historical toxic gas components, the historical gas treatment measures, and the treatment relationship. In the embodiment of the present application, the electronic device may acquire the historical toxic gas components, the historical gas treatment measures, and the treatment relationship between the historical toxic gas components and the historical gas treatment measures from local storage, or from other devices, or may acquire the historical toxic gas components, the historical gas treatment measures, and the treatment relationship between the historical toxic gas components and the historical gas treatment measures input by the user, which is not limited in the embodiment of the present application.

For the embodiment of the present application, when there is no person in the target environment, it is necessary to treat the current toxic gas in the target environment, when there are at least two current toxic gas components, it is necessary to determine the current toxic gas component to be treated first; different toxic gases have different impacts on the environment, and it is necessary to determine the current danger levels corresponding to each current toxic gas component from the preset danger levels according to the relationship between the preset toxic gas component and the preset danger level; the greater the current danger level, the greater the impact on the target environment. The impact of the current toxic gas components on the target environment also includes the current toxic gas concentrations; since the current toxic gas components may exist at each detection position, average values of the current toxic gas components at each detection position may be determined as the component concentrations corresponding to the current toxic gas components, and the current toxic gas component with the greatest impact on the target environment, i.e., the current emergency toxic gas component, is determined jointly through the current danger levels and component concentrations corresponding to each current toxic gas component; different toxic gas components may correspond to different treatment measures.

For the embodiment of the present application, the average values of the current toxic gas components at each detection position may be determined as the component concentrations corresponding to the current toxic gas components; for example, the current toxic gas concentration of current toxic gas component A at the detection position one is 14 mg/m³, and the current toxic gas concentration at the detection position two is 28 mg/m³, then the component concentration of the current toxic gas component A is 21 mg/m³; the current toxic gas concentration of the current toxic gas component B at the detection position one is 23 mg/m³, and the current toxic gas concentration at the detection position two is 29 mg/m³, then the component concentration of the current toxic gas component B is 26 mg/m³.

Further, the weight value of each current toxic gas component may be determined according to the danger level, the component concentration, and preset weight corresponding to each current toxic gas component, and the current toxic gas component with the maximum weight value is determined as the current emergency toxic gas component.

For the embodiment of the present application, the treatment measure may be condensation to accelerate the treatment of the current toxic gas, and the current gas treatment measure for the current emergency toxic gas component is determined from the historical gas treatment measures based on the historical toxic gas components and the treatment relationship between the historical toxic gas components and the historical gas treatment measures; for example, the historical gas treatment measure corresponding to the historical toxic gas component A is measure a, the historical gas treatment measure corresponding to the historical toxic gas component B is measure b, and when the current toxic gas component A is the same as the historical toxic gas component A, then the current gas treatment measure for the current toxic gas component A is measure b.

Another possible implementation for determining the current gas treatment measure is to train an original model based on the historical toxic gas components and the historical gas treatment measures to obtain a trained current gas treatment measure recognition model, and inputting the current emergency toxic gas component into the current gas treatment measure recognition model for recognition to obtain the current gas treatment measure; in the embodiment of the present application, since the current gas treatment measure recognition model calculates quickly, efficiency of determining the current gas treatment measure is improved.

For the embodiment of the present application, when there are at least two current toxic gas components, the current toxic gas component requiring emergency treatment is determined through the danger level of the current toxic gas component and the current toxic gas concentration, and the current treatment measure is accurately determined according to the historical gas treatment measures corresponding to the historical toxic gas components, thereby reducing harm of toxic gases.

The above embodiment introduces the method for toxic gas detection from the perspective of the method flow, and the following embodiment introduces an equipment for toxic gas detection from the perspective of a virtual module or a virtual unit, which is detailed in the following embodiment.

An embodiment of the present application provides an equipment for toxic gas detection. referring to FIG. 2, the equipment 20 for toxic gas detection may specifically include: a first acquisition module 21, a first determination module 22, and a second determination module 23, wherein,
the first acquisition module 21 is configured to acquire the current toxic gas components in the target environment, the current toxic gas concentrations corresponding to the current toxic gas components at each detection position, and the first position identification information corresponding to each detection position;
the first determination module 22 is configured to determine the current diffusion position based on the current toxic gas components and the current toxic gas concentrations and the first position identification information corresponding to each detection position; and
the second determination module 23 is configured to acquire second position identification information corresponding to the person, and determine and output the current escape route based on the current diffusion position and the second position identification information.

In a possible implementation of the embodiment of the present application, the equipment 20 further includes: a first judging module and a second judging module, wherein,
the first judging module is configured to judge whether the target environment is the dangerous area based on the current toxic gas concentrations corresponding to each detection position;
the second judging module is configured to, when the target environment is the dangerous area, acquire environmental images corresponding to the target environment, and judge whether there is the person in the target environment based on the environmental images.

In another possible implementation of the embodiment of the present application, the first judging module, when judging whether the target environment is the dangerous area based on the current toxic gas concentrations corresponding to each detection position, is specifically configured to:
acquire the first relationship curve corresponding to each detection position, wherein the first relationship curve is configured to characterize the relationship between the historical toxic gas concentrations and the historical time corresponding to each detection position within the historical time;
determine the environmental concentration thresholds corresponding to each detection position based on the first relationship curve;
when the current toxic gas concentration is greater than the environmental concentration threshold, determine the detection position corresponding to the current toxic gas concentration as the dangerous position; and
when the number of the dangerous positions is greater than the threshold, the target environment is the dangerous area.

In another possible implementation of the embodiment of the present application, the first determination module 22, when determining the current diffusion position based on the current toxic gas components, and the current toxic gas concentrations and the first position identification information corresponding to each detection position, is specifically configured to:
determine the current diffusion direction corresponding to the current toxic gas components based on the current toxic gas components, the preset toxic gas component, the preset diffusion direction, and the diffusion relationship between the preset toxic gas component and the preset diffusion direction;
determine the highest current toxic gas concentration from the current toxic gas concentrations corresponding to each detection position, and determine the detection position corresponding to the highest current toxic gas concentration as the first detection position; and
determine the current diffusion position based on the current diffusion direction and the first position identification information corresponding to the first detection position.

In another possible implementation of the embodiment of the present application, the second determination module 23, when determining the current escape route based on the current diffusion position and the second position identification information, is specifically configured to
determine the diffusion range based on the current toxic gas concentration and the first position identification information corresponding to each detection position;
determine the current escape route based on the current diffusion position, the second position identification information, and the diffusion range.

In another possible implementation of the embodiment of the present application, the second determination module 23, when determining the diffusion range based on the current toxic gas concentrations and the first position identification information corresponding to each detection position, is specifically configured to
determine the current minimum toxic gas concentration from the current toxic gas concentrations corresponding to each detection position based on the environmental concentration thresholds corresponding to each detection position, and determine the detection position corresponding to the current minimum toxic gas concentration as the second detection position, where the current minimum toxic gas concentration is higher than the environmental concentration threshold corresponding to the second detection position;
determine the diffusion distance based on the first position identification information corresponding to the first detection position and the first position identification information corresponding to the second detection position; and
determine the diffusion range based on the first position identification information corresponding to the first detection position, the first position identification information corresponding to the second detection position, and the diffusion distance.

In another possible implementation of the embodiment of the present application, when the current toxic gas component is at least in a number of two, the equipment 20 further includes: a fourth determination module, a fifth determination module, a sixth determination module, a third acquisition module, and a seventh determination module, wherein,
the fourth determination module is configured to, when there is no person in the target environment, determine the current danger levels corresponding to each current toxic gas component based on each current toxic gas component, the preset toxic gas component, the preset danger level, and the relationship between the preset toxic gas component and the preset danger level;
the fifth determination module is configured to determine the component concentrations corresponding to each current toxic gas component based on the current toxic gas concentrations corresponding to each current toxic gas component at each detection position;
the sixth determination module is configured to determine the current emergency toxic gas component from each current toxic gas component based on the current danger levels and the component concentrations corresponding to each current toxic gas component;
the third acquisition module is configured to acquire the historical toxic gas components, the historical gas treatment measures, and the treatment relationship between the historical toxic gas components and the historical gas treatment measures; and
the seventh determination module is configured to determine the current gas treatment measures based on the current emergency toxic gas components, the historical toxic gas components, the historical gas treatment measures, and the treatment relationship.

The embodiment of the present application provides the equipment for toxic gas detection, compared with the related art, in the embodiment of the present application, by acquiring the current toxic gas components in the target environment, the current toxic gas concentrations corresponding to the current toxic gas components at each detection position, and the first position identification information corresponding to each detection position respectively; the current diffusion position is determined based on the current toxic gas components, and the current toxic gas concentrations and the first position identification information corresponding to each detection position; when determining the escape route, it is necessary to avoid the diffusion position of the toxic gas; the second position identification information corresponding to the person is acquired; and the current escape route is accurately determined and output based on the current diffusion position and the second position identification information to guide the person in the target environment to escape, and timely determine the escape route of the person in the target environment in case of toxic gas leakage.

Those skilled in the art can clearly understand that, for the convenience and conciseness of description, specific working processes of the equipment for toxic gas detection can refer to the corresponding processes in the above-mentioned method embodiments, which will not be repeated here.

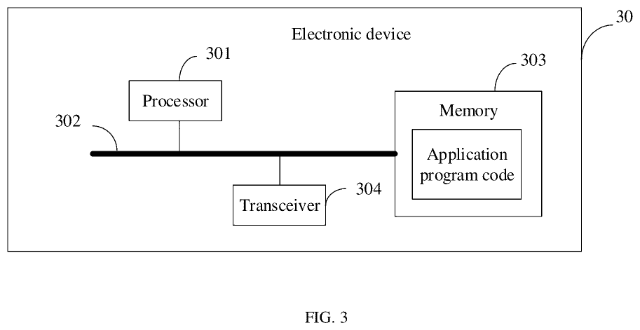

The embodiment of the present application provides an electronic device. Referring to FIG. 3, the electronic device 30 includes: a processor 301 and a memory 303. Wherein, the processor 301 is connected to the memory 303, such as via a bus 302. Optionally, the electronic device 30 may further include a transceiver 304. It should be noted that in practice, a number of transceivers 304 is not limited to one, and a structure of the electronic device 30 does not constitute a limitation to the embodiment of the present application.

The processor 301 may be a Central Processing Unit (CPU), a general-purpose processor, a Digital Signal Processor (DSP), an Application Specific Integrated Circuit (ASIC), a Field Programmable Gate Array (FPGA), or other programmable logic devices, transistor logic devices, hardware components, or any combination thereof. It may implement or execute various exemplary logical blocks, modules, and circuits described in connection with the disclosure of the present application. The processor 301 may also be a combination for implementing computing functions, such as a combination including one or more microprocessors, a combination of the DSP and a microprocessor, etc.

The bus 302 may include a path for transmitting information between the above components. The bus 302 may be a Peripheral Component Interconnect (PCI) bus, an Extended Industry Standard Architecture (EISA) bus, etc. The bus 302 may be divided into an address bus, a data bus, a control bus, etc. For ease of illustration, only one bold line is shown in FIG. 3, but does not indicate that there is only one bus or one type of bus.

The memory 303 may be a Read Only Memory (ROM) or a static storage device of other components that can store static information and instructions, a Random Access Memory (RAM) or a dynamic storage device of other components that can store information and instructions, and may also be an Electrically Erasable Programmable Read Only Memory (EEPROM), a Compact Disc Read Only Memory (CD-ROM) or other optical disc storage (including compact discs, laser discs, optical discs, digital versatile discs, Blu-ray discs, etc.), magnetic disk storage media or other magnetic storage devices, or any other medium that can be used to carry or store desired program code in the form of instructions or data structures and can be accessed by a computer, but is not limited thereto.

The memory 303 is configured to store application program code for executing the solutions of the present application, and is controlled to execute by the processor 301. The processor 301 is configured to execute the application program code stored in the memory 303 to implement the content shown in the above-mentioned method embodiment.

Wherein, electronic devices include, but are not limited to mobile terminals such as mobile phones, notebook computers, digital broadcast receivers, personal digital assistants (PDA), tablet computers (PAD), portable multimedia players (PMP), in-vehicle terminals (e.g., in-vehicle navigation terminals, etc.), and fixed terminals such as digital TVs, desktop computers, etc. It can also be a server, etc. The electronic device shown in FIG. 3 is only one example and should not impose any limitation on the functionality and scope of use of the embodiment of the present application.

The embodiment of the present application provides a computer-readable storage medium, where a computer program is stored on the computer-readable storage medium, and when the computer program runs on the computer, the computer can execute the corresponding content in the above-mentioned method embodiment. Compared with the related art, in the embodiment of the present application, by acquiring the current toxic gas components in the target environment, the current toxic gas concentrations corresponding to the current toxic gas components at each detection position, and the first position identification information corresponding to each detection position respectively; the current diffusion position is determined based on the current toxic gas components, and the current toxic gas concentrations and the first position identification information corresponding to each detection position; when determining the escape route, it is necessary to avoid the diffusion position of the toxic gas; the second position identification information corresponding to the person is acquired; and the current escape route is accurately determined and output based on the current diffusion position and the second position identification information to guide the person in the target environment to escape, and timely determine the escape route of the person in the target environment in case of toxic gas leakage.

It should be understood that, although various steps in the flowchart of the figure are shown sequentially as indicated by the arrows, these steps are not necessarily performed sequentially in the order indicated by the arrows. Unless otherwise explicitly stated in this document, there is no strict sequential order restriction on the performance of these steps, and they may be performed in other orders. Furthermore, at least some of the steps in the flowchart of the accompanying drawings may include a plurality of sub-steps or a plurality of phases. These sub-steps or phases are not necessarily performed and completed at the same time, but may be performed at different times; their execution order is also not necessarily carried out sequentially, but may be performed alternately or in turns with at least part of other steps, or the sub-steps or phases of other steps.

The foregoing is merely part of the embodiments of the present application. It should be noted that, for those skilled in the art, various modifications and refinements may be made without departing from the principles of the present application, and such modifications and refinements shall also be regarded as falling within the protection scope of the present application.

What is claimed is:

1. A method for toxic gas detection, comprising:
acquiring current toxic gas components in a target environment, a current toxic gas concentration of each of the current toxic gas components at each detection position, and first position identification information at each detection position,
determining whether the target environment is a dangerous area based on the current toxic gas concentration at each detection position,
acquiring an environmental image of the target environment, when the target environment is the dangerous area, and determining whether there is a person in the target environment based on the environmental image, when so, determining a current diffusion position based on the current toxic gas components, and the current toxic gas concentration and the first position identification information at each detection position,
acquiring second position identification information of the person, and determining and outputting a current escape route based on the current diffusion position and the second position identification information,
wherein determining the current escape route based on the current diffusion position and the second position identification information comprises:
determining a diffusion range based on the current toxic gas concentration and the first position identification information at each detection position, and
determining the current escape route based on the current diffusion position, the second position identification information, and the diffusion range,
wherein determining the diffusion range based on the current toxic gas concentration and the first position identification information at each detection position comprises:
determining a current minimum toxic gas concentration from the current toxic gas concentrations at each detection position based on environmental concentration thresholds at the detection positions, and defining a detection position with the current minimum toxic gas concentration as a second detection position, wherein the current minimum toxic gas concentration is greater than a respective one of the environmental concentration thresholds at the second detection position, and the current minimum toxic gas concentration is a concentration with a smallest difference from a respective one of the environmental concentration thresholds,
determining a diffusion distance based on the first position identification information at a first detection position and the first position identification information at the second detection position, wherein the first detection position is a detection position with a maximum toxic gas concentration among the current toxic gas concentrations at the detection positions, and
determining the diffusion range based on the first position identification information at the first detection position, the first position identification information at the second detection position, and the diffusion distance, wherein the first position identification information at the first detection position is defined as a center point, a circle is drawn with a distance between the first detection position and the second detection position as a radius, and a range covered by the circle is a current diffusion range,
wherein determining the current diffusion position based on the current toxic gas components, and the current toxic gas concentration and the first position identification information at each detection position comprises:
determining a current diffusion orientation of the current toxic gas components based on the current toxic gas components, preset toxic gas components, a preset diffusion orientation, and a diffusion relationship between the preset toxic gas components and the preset diffusion orientation,
determining a current maximum toxic gas concentration among the current toxic gas concentrations at the detection positions, and defining a detection position of the detection positions with the current maximum toxic gas concentration as the first detection position, and
determining the current diffusion position based on the current diffusion orientation and the first position identification information at the first detection position.

2. The method according to claim 1, wherein determining whether the target environment is the dangerous area based on the current toxic gas concentration at each detection position comprises:
acquiring a first relationship curve corresponding to each detection position, the first relationship curve is configured to characterize a relationship between a historical toxic gas concentration and historical time at each detection position within a historical duration,
determining a respective one of the environmental concentration thresholds at each detection position based on the first relationship curve,
defining a detection position with the current toxic gas concentration as a dangerous position when the current toxic gas concentration is greater than the respective one of the environmental concentration thresholds, and
defining the target environment as the dangerous area when a number of dangerous positions is greater than a threshold.

3. The method according to claim 1, wherein when at least two current toxic gas components are present, the method further comprises:
determining a current danger level of each of the current toxic gas components based on the current toxic gas components, the preset toxic gas components, a preset danger level, and relationships between the preset toxic gas components and the preset danger level when there is no person in the target environment,
determining a component concentration of each of the current toxic gas components based on the current toxic gas concentration of each of the current toxic gas components at each detection position,
determining a current emergency toxic gas component from the current toxic gas components based on the current danger level and the component concentration of each of the current toxic gas components,
acquiring historical toxic gas components, historical gas treatment measures, and a treatment relationship between the historical toxic gas components and the historical gas treatment measures, and
determining a current gas treatment measure based on the current emergency toxic gas component, the historical toxic gas components, the historical gas treatment measures, and the treatment relationship between the historical toxic gas components and the historical gas treatment measures.

4. An equipment for toxic gas detection, comprising:
a first acquisition module configured to acquire current toxic gas components in a target environment, a current toxic gas concentration of each of the current toxic gas components at each detection position, and first position identification information at each detection position,
a first determination module configured to determine a current diffusion position based on the current toxic gas components, and the current toxic gas concentration and the first position identification information at each detection position, and
a second determination module configured to acquire second position identification information of a person, and to determine and output a current escape route based on the current diffusion position and the second position identification information,
wherein the equipment further comprises a first judging module and a second judging module,
the first judging module is configured to determine whether the target environment is a dangerous area based on the current toxic gas concentration at each detection position, and
the second judging module is configured to acquire an environmental image of the target environment when the target environment is the dangerous area, to determine whether there is a person in the target environment based on the environmental image, and when so, to trigger the first determination module,
wherein when determining the current escape route based on the current diffusion position and the second position identification information, the second determination module is configured to
determine a diffusion range based on the current toxic gas concentration and the first position identification information at each detection position, and
determine the current escape route based on the current diffusion position, the second position identification information, and the diffusion range;
wherein when determining the diffusion range based on the current toxic gas concentration and the first position identification information at each detection position, the second determination module is configured to
determine a current minimum toxic gas concentration from the current toxic gas concentrations at each detection position based on environmental concentration thresholds at the detection positions, and define a detection position with the current minimum toxic gas concentration as a second detection position, wherein the current minimum toxic gas concentration is greater than a respective one of the environmental concentration thresholds at the second detection position, and the current minimum toxic gas concentration is a concentration with a smallest difference from a respective one of the environmental concentration thresholds,
determine a diffusion distance based on the first position identification information at a first detection position and the first position identification information at the second detection position, wherein the first detection position is a detection position with a maximum toxic gas concentration among the current toxic gas concentrations at the detection positions, and
determine the diffusion range based on the first position identification information at the first detection position, the first position identification information at the second detection position, and the diffusion distance, wherein the first position identification information at the first detection position is defined as a center point, a circle is drawn with a distance between the first detection position and the second detection position as a radius, and a range covered by the circle is a current diffusion range;
wherein when determining the current diffusion position based on the current toxic gas components, and the current toxic gas concentration and the first position identification information at each detection position, the first determination module is configured to
determine a current diffusion orientation of the current toxic gas components based on the current toxic gas components, preset toxic gas components, a preset diffusion orientation, and a diffusion relationship between the preset toxic gas components and the preset diffusion orientation,
determine a current maximum toxic gas concentration among the current toxic gas concentrations at the detection positions, and define a detection position of the detection positions with the current maximum toxic gas concentration as the first detection position; and
determine the current diffusion position based on the current diffusion orientation and the first position identification information at the first detection position.

5. The equipment according to claim 4, wherein the first judging module is further configured to:
acquire a first relationship curve corresponding to each detection position, the first relationship curve is configured to characterize a relationship between a historical toxic gas concentration and historical time at each detection position within a historical duration,
determine a respective one of the environmental concentration thresholds at each detection position based on the first relationship curve,
define a detection position with the current toxic gas concentration as a dangerous position when the current toxic gas concentration is greater than the respective one of the environmental concentration thresholds, and
define the target environment as the dangerous area when a number of dangerous positions is greater than a threshold.

6. The equipment according to claim 4, wherein when the current toxic gas components are at least in the number of two, the equipment further comprises a fourth determination module, a fifth determination module, a sixth determination module, a third acquisition module, and a seventh determination module,
the fourth determination module is configured to determine a current danger level of each of the current toxic gas components based on the current toxic gas components, the preset toxic gas components, a preset danger level, and relationships between the preset toxic gas components and the preset danger level when there is no person in the target environment,
the fifth determination module is configured to determine a component concentration of each of the current toxic gas components based on the respective current toxic gas concentration of each of the current toxic gas components at each detection position,
the sixth determination module is configured to determine a current emergency toxic gas component from the current toxic gas components based on the current danger level and the component concentration of each of the current toxic gas components,
the third acquisition module is configured to acquire historical toxic gas components, historical gas treatment measures, and a treatment relationship between the historical toxic gas components and the historical gas treatment measures, and
the seventh determination module is configured to determine a current gas treatment measure based on the current emergency toxic gas component, the historical toxic gas components, the historical gas treatment measures, and the treatment relationship between the historical toxic gas components and the historical gas treatment measures.

7. An electronic device, comprising:
one or more processors,
a memory, and
one or more application programs, wherein the one or more application programs are stored in the memory and configured to be executed by the one or more processors to implement the method for toxic gas detection according to claim 1.

8. A computer-readable storage medium comprising a computer program stored thereon, wherein when the computer program is executed by a processor, the method for toxic gas detection according to claim 1 is implemented.
